(19) United States
(12) Patent Application Publication
(10) Pub. No.: US 2010/0129910 A1
(43) Pub. Date: May 27, 2010

(54) Cell matrix related compositions and their use for generating embryoid bodies

(76) Inventors: Denis Evseenko, Pasadena, CA (US); Gay Crooks, Sherman Oaks, CA (US)

Correspondence Address: STAHL LAW FIRM, 2 MEADOWSWEET LANE, SAN CARLOS, CA, 94070, US

(21) Appl. No.: 12/459,582

(22) Filed: Jul. 2, 2009

Related U.S. Application Data
(60) Provisional application No. 61/133,799, filed on Jul. 2, 2008.

(51) Int. Cl. C12N 5/02; C12N 5/00

(52) U.S. Cl. 435/383; 435/404; 435/325

(57) ABSTRACT

The present invention relates to compositions and methods for the generation of embryoid bodies from embryonic stem cells, and for aggregation of ES cells for establishing ES cell colonies from small numbers of cells. Compositions of the invention, in certain embodiments, comprise embryonic stem cells, an extracellular matrix molecule, and a molecule capable of cross linking. The present invention also relates to methods for embryoid body formation using a composition of the invention and to cells and tissues isolated or derived from an embryoid body generated with a composition or method of the invention.

This application claims the benefit of U.S. Provisional Application No. 61/133,799, filed Jul. 2, 2008, which is incorporated herein by reference in its entirety.

The research was made possible by a grant from the California Institute for Regenerative Medicine (Grant Number RC1-00108-1).

### 1.0 FIELD OF THE INVENTION

The invention relates to compositions and methods to promote aggregation of Embryonic stem (ES) cells and/or to generate Embryoid Bodies (EB) from aggregated ES cells. The invention also relates to establishing an ES cell culture from one or more ES cells after processing of ES cells, for example, following cell sorting or selection procedures.

### 2.0 BACKGROUND

ES cells are primarily derived from the inner cell mass of blastocysts formed during mammalian (including human, non-human primate and murine) development. ES cells can be grown in culture and they may be capable of generating three of the germ lines that make up the developing embryo (in other words, the ectoderm, the mesoderm and the endoderm). Further differentiation into a diverse range of cells and/or tissues, including mature cells and/or tissues, may also result.

Human ES cells (hESC) have multiple uses as research tools and are useful for clinical applications, including cell and tissue therapy and in vitro drug testing. The use of hESC for the generation of tissue for either experimental or therapeutic purposes requires different stages of cell culture. For example, a prolonged expansion (or “passaging”) of ES cells may be required and during this stage ES cells are maintained in an undifferentiated and pluripotent state. During another stage, culture conditions are changed to induce differentiation of ES cells into more mature cells and/or tissues. Such a differentiation stage or phase may be accomplished through the formation of EB, which are three-dimensional cell aggregates within which multiple tissue types may be produced at different stages of development. An EB should recapitulate some or all of the conditions found in the environment of an embryo.

It is believed that intercellular contact facilitates the maintenance of hESC cells in an undifferentiated and viable state. hESC may be cultured on layers of human or murine feeder cells (for example, mouse embryonic fibroblasts (MEFs)), or in a “feeder-free” culture system using culture plates with a coated surface (for example, with extracellular matrix) onto which hESC adhere in tightly aggregated colonies. To prevent the loss of cell-cell contact during passaging of cultured hESC, cells are moved from one plate to another as clumps of partially digested or mechanically dislodged colonies.

The generation of human EBs (hEBs) involves transferring hESC from their adherent layers into non-contact, suspension cultures. Generation of hEBs can be attempted in two ways. One method involves removing clumps of partially digested ES cell colonies from MEFs and re-plating then into non-contact cultures (classic method). This method requires large numbers of hESC, results in considerable cell death, and cannot be well standardized and quantitated. Another method involves complete digestion of ES cells to generate single cell suspensions, re-aggregation of the ES cells by centrifugation, and plating the re-formed clumps in non-contact culture (spin method). This method requires components that result in contamination of the hEBs, for example, the presence of feeder cells or protein mixtures derived from animal extracellular matrix.

A method of EB formation that consistently and efficiently produces high quality EBs, without the need for contaminating cells or non-human proteins, would greatly assist in the application of hESC (or other pluripotent cell types), and the cells and tissues derived therefrom, in research and therapy. The invention relates to compositions and methods to re-aggregate human ES cells without the need for contaminating cells or xenogeneic proteins, so that they can better generate ES cell cultures and/or embryoid bodies efficiently and consistently.

### 3.0 SUMMARY OF THE INVENTION

The present invention relates to compositions and methods for EB formation. In certain embodiments, compositions of the invention comprise ES cells and an extracellular matrix component, laminin, and a component capable of cross-linking said extracellular matrix component, nidogen. The ES cells and the remaining components of a composition of the invention are preferably human, and less preferably from another species. Cells and other components may be from different species.

A composition of the invention, in certain embodiments, can be used to promote aggregation of ES cells, for example, to culture ES cells. Establishing an ES cell culture from a small number of ES cells, including a single cell suspension, may therefore be promoted with a composition of the invention. A small ES cell population comprising one, two, three, four, five, or more ES cells, may be obtained through cell sorting (e.g., FACS) or another cell processing method, or from a blastocyst, a morula, or another tissue, for example, an embryonic, fetal, postnatal, adult or any other tissue.

A composition of the current invention, in certain embodiments, comprises laminin and nidogen. In certain embodiments, a composition of the invention comprises fibronectin. In certain other embodiments, a composition of the invention further comprises a synthetic tissue culture medium, a buffer, a protein, a glycoprotein, an extracellular matrix component, an extracellular matrix protein fragment, a collagen, a component of a serum, a sugar, glucose, a salt, a dye, a vitamin, a metal, a growth factor, a conditioned medium, or any other component useful for the maintenance of ES cells and/or an EB in culture, or a combination of several of these components, for example, one, two, three, four, five, six, or more of these components. In certain other embodiments, a composition of the invention further comprises a support that facilitates isolation of ES cells and/or EBs, for example, a matrix, a scaffold, beads, a tissue culture dish, or any other support.

In certain other embodiments, a method of the invention comprises inducing or facilitating EB formation in the presence of a composition of the invention, for example, in cell culture or cell suspension. A composition of the invention is used in a method of the invention in an amount sufficient to result in desirable EB formation, for example, by measuring an increase in life span of the EBs, by observing an improved formation of all three germ layers (in other words, an ectoderm layer, a mesoderm layer, and an endoderm layer), by observing a higher total cell number per EB (for example, by disassociating EBs and counting cells using known methods), and/or by observing a higher number of tissue specific progenitor cells (for example, hematoendothelial progenitor cells) in the EBs.

Desirable EB formation can be measured, in certain embodiments, by comparing one or more of the above parameters in EBs generated using a composition or method of the invention versus the same parameters in EBs using the classic method and/or the spin method. Desirable EB formation, in certain embodiments, results in an improvement of at least 10 percent, at least 20 percent, at least 30 percent, at least 40 percent or at least 50 percent in one, two, or three of the above parameters (in other words, improved formation of three germ layers, higher total cell number, higher number of tissue specific progenitor cells).

In certain other embodiments, the invention provides EBs generated using a composition or method of the invention, and cells and/or tissues obtained from or derived from an EB generated using a composition or method of the invention.

### 4.0 BRIEF DESCRIPTION OF THE DRAWINGS

FIG. 1: EB formation with human ES cells (hES cells) in the presence of laminin and nidogen is shown. A. 3000 hES cells are shown following centrifugation (left) and again 4 hours later when the cells have aggregated and an EB has formed. B. hES cells of different numbers (10,000; 3,000; 1,000; 300; 100; 30) were incubated with laminin and nidogen.

FIG. 2: Expression of ECM proteins, mRNA and their receptors in undifferentiated hESC colonies. A. Immunofluorescence staining of hESC and MEFs for expression of collagen type IV; laminin; nidogen, heparan sulphate proteoglycans (HSPG) (all green). Polyclonal antibodies recognize both mouse and human ECM proteins. Staining using the secondary antibodies alone served as controls. F-actin staining (red) visualizes the cell cytoskeleton in all panels. DAPI-staining (blue) was performed to show cell nuclei. Scale bar equals 40 μm. B. RT-PCR analysis (35 PCR cycles) indicates that hESC express transcripts for the laminin chains α5, β1, γ1 and γ3; nidogen-1; and also COL4α1, COL4α2, COL4α5 and COL4α6 genes encoding the collagen IV polypeptides. Panel C. shows co-expression of the pluripotency marker SSEA-4 (magenta) with a high co-expression of integrins α1 (green) and α6 (red) in the same cell shown in the bright field panel on far left. D. This expression pattern was demonstrated with FACS for more than 90% of analyzed hESC cells.

FIG. 3: Induction of hESC aggregation by Matrigel, laminins-111 and -511, collagen-IV and nidogen-1. A. Efficiency of cell aggregation under different culture condition can be expressed using semi-quantitative scoring system: “−” represents no cell aggregation, “+++” indicates that 40-60% of cells in the well aggregated and formed EB, while others remain single cells, and “+++++” shows that most of the cells in the well aggregated and formed EB after 24 hour in culture. B. Appearance of 3000 hESC immediately following by centrifugation in low contact plates. C. In absence of MEFs or ECM during centrifugation, no cell aggregation and EB is detected. D. Robust cell aggregation is observed within 4 to 8 hours of centrifugation (shown on the picture) when laminin-nidogen complex is added to a suspension of 3,000 hESC (in the absence of MEFs), with completed formation of EBs observed after 24 hours in culture. Magnification: ×40. E. Effects of different ECM inducers on hESC aggregation. Final concentrations of the proteins used for the aggregation can be found in the Materials and Methods section.

FIG. 4: Generation of ectodermal, mesodermal and endodermal derivatives in the “LN-EB” system. “LN-EBs” were generated with the laminin-nidogen complex and in the presence of media designed for specific germ layer differentiation. A. For induction of ectodermal derivatives, serum-free media was supplemented with Wnt3a (10 ng/ml); B. Addition of BMP4, FGF2, VEGF (all 10 ng/ml) to serum-free medium induced formation of endodermal and mesodermal derivatives. C. Addition of 10% FCS, with no exogenous growth factors, induced synchronous generation of the all three germ layers. D. Standard EBs cultured in medium supplemented with 10% FCS. E. Control EBs cultured in serum-free media with no exogenous growth factors. F. No primary antibody control of immunostaining. Immunohistochemical detection of ectoderm, mesoderm and endoderm formation was assessed using antibodies for nestin (left column), CD34 (middle column) and alpha-fetoprotein (right column) respectively. Antigen expression is shown in brown color. Nuclei are stained with hematoxylin (blue). Magnification: 200×.

FIG. 5: Generation of hematoendothelial progenitor cells in the “LN-EB” system. To assess efficiency of hematoendothelial progenitor cell formation in different culture conditions, “LN-EBs” were cultured either in control medium (serum-free, no growth factors), media supplemented with 10% FCS or “BVF” medium (serum free with BMP4, FGF2 and VEGF (all 10 ng/ml); “Standard EBs” were cultured in medium supplemented with 10% FCS and no growth factors. EBs were dissociated into a single cell suspension at day 8, 15 and 23 of culture and labeled with CD34, CD31, KDR and BB9 antibodies. Three major populations (A): CD34^{bright}, CD31^{bright}, KDR^{bright}, BB9^{dim}, (B): CD34^{dim}, CD31^{dim}, KDR_{neg}, BB9_{neg}, and (C): CD34^{dim}, CD31^{dim}, KDR_{neg}, BB9^{bright} were identified with FACS. Absolute number of cells were determined for each of these populations and expressed per 1,000 hESC plated at initial stage of EB formation. D. Location of the populations on representative FACS plots from LN-EB cultured for 15 days in presence of 10% FCS with no growth factors added. Population A is shown in light blue, populations B and C in yellow and green respectively. Results are expressed as mean±SD of five independent experiments. For hematoendothelial progenitor generation studies one-way ANOVA was applied followed by Student-Newman-Keul's test. P<0.05 was considered to be significant.

FIG. 6: Production of ECM proteins in LN-EBs. The ECM proteins are deposited as massive protein islands within the EBs (A), or diffusely distributed within the cellular compartments (B, C, D). Frozen sections of LN-EBs were prepared on day 15 of culture and exposed to primary polyclonal antibodies against collagen IV, laminin and nidogen 1, respectively, followed by the secondary antibodies conjugated with Cy3 (red color). Nuclei were visualized with Dapi staining (blue).

### 5.0 DETAILED DESCRIPTION OF THE INVENTION

The present invention relates to compositions and methods for EB formation. In certain embodiments, compositions of the invention comprise ES cells and an extracellular matrix component, for example laminin, and a component capable of cross-linking said extracellular matrix component, for example nidogen. In certain embodiments, a composition of the invention comprises fibronectin. Laminin, nidogen and/or fibronectin of a composition of the invention, in certain embodiments, may be of any species, for example, human, mouse, rat, bovine, or any other species. Laminin, nidogen and/or fibronectin of a composition of the invention, in certain embodiments, may be generated in any way, for example, through expression in cells, for example, in ES cells, fibroblasts, or any other cell type, or through expression in an EB, or through any other means of synthesis, including synthetic methods. In certain preferred embodiments, a composition of the invention does not comprise an ES cell.

A medium of the current invention in certain embodiments contains each of laminin and nidogen at a concentration that may be independently selected from 0.005-5.0 mg/ml, or 0.01-2.5 mg/ml, or 0.02-1.25 mg/ml, or 0.04-0.5 mg/ml, or 0.08-0.15 mg/ml. A composition of the invention comprising fibronectin, in certain embodiments, may comprise a lower concentration of laminin and/or nidogen when compare to a composition of the invention without fibronectin, for example, 10 percent lower, or 20 percent lower, or 30 percent lower, or 40 percent lower, or 50 percent lower. The laminin, nidogen and/or fibronectin of a composition of the invention, in certain embodiments, may be independently selected from a naturally occurring composition, a synthetic composition, and/or a protein fragment of laminin and/or nidogen. The ES cells and the remaining components of a composition of the invention are preferably human, and less preferably from another species. Cells and other components may be from different species.

In certain other embodiments, a composition of the invention further comprises a synthetic tissue culture medium, a buffer, a protein, a glycoprotein, an extracellular matrix component, an extracellular matrix protein fragment, a collagen, a component of a serum, a sugar, glucose, a salt, a dye, a vitamin, a metal, a growth factor, a conditioned medium, or any other component useful for the maintenance of ES cells and/or an EB in culture, or a combination of several of these components, for example, one, two, three, four, five, six, or more of these components. In certain other embodiments, a composition of the invention further comprises a support that facilitates isolation of ES cells and/or EBs, for example, a matrix, a scaffold, beads, a tissue culture dish, or any other support.

In certain other embodiments, a composition of the invention does not include a non-human protein, a xenogeneic protein, a cell type other than an ES cell, a complete serum (meaning that no complete serum was added to the medium). In certain other embodiments, a composition of the invention includes a complete serum in an amount that is insufficient to sustain ES cells. In certain other embodiments, a composition of the invention includes an incomplete serum, meaning a serum that is not sufficiently complete to sustain ES cells, for example, a serum that is 90 percent complete, or 75 percent, or 50 percent, or 25 percent, or 10 percent. In certain other embodiments, a composition of the invention is conditioned, for example, by fibroblasts, MEF's, or any cell type capable of sustaining ES cells.

In certain other embodiments, a method of the invention comprises inducing or facilitating aggregation of ES cells and/or establishment of ES cell culture from a sample of ES cells, for example, from 1, 2, 3, 4, 5, 6, 7, 8, 9, 10, 15, 20, 30, 50, 100 ES cells. In certain other embodiments, a method of the invention comprises inducing or facilitating EB formation in the presence of a composition of the invention, for example, in cell culture or cell suspension. A composition of the invention is used in a method of the invention in an amount sufficient to result in desirable EB formation, for example, by measuring an increase in life span of the EBs, by observing an improved formation of all three germ layers (in other words, an ectoderm layer, a mesoderm layer, and an endoderm layer), by observing a higher total cell number per EB (for example, by disassociating EBs and counting cells using known methods), and/or by observing a higher number of tissue specific progenitor cells (for example, hematoendothelial progenitor cells) in the EBs.

Background on ES cells, including the isolation, culturing and use of ES cells, is also described in U.S. Patent Application Nos. 20060030040; 20060040383; 20060206953; 20060252150; and in U.S. Pat. Nos. 5,166,065; 5,690,926; 5,843,780; 6,576,464; 6,875,607; 6,875,608; 6,921,665; 7,029,913; 7,041,438; 7,112,437; 7,186,883; 7,413,904; 7,449,334, all of which are incorporated herein by reference for all purposes, and a composition and a method of the current invention may be applied to an ES cell described in any of these references. Background on EBs is also described in U.S. Patent Application Nos. 20030175954; 20040023376; 20040096967; 20050186182; 20070015210; 20080019952; and in U.S. Pat. No. 7,220,584, all of which are incorporated herein by reference for all purposes.

The present invention is further illustrated by the following examples, which are not intended to be limiting in any way whatsoever.

### Examples

### Example 1

### EB Formation in the Presence of Laminin and Nidogen

1.1 Background

We have developed a simple method for the efficient generation of human EBs(hEB) from defined numbers of hES cells. The classical method of hEB formation is to plate clumps of partially digested hESC into non-contact tissue culture plates. The suspended clumps then spontaneously differentiate into hEB that vary in size and morphology. A more recent method, called “spin EBs” completely digests hES cells into single cell suspensions that can be counted and manipulated, and then uses centrifugation to re-aggregate hES cells for subsequent differentiation into hEB.

1.2. Laminin and Nidogen Facilitate EB Formation from hES Cell Suspensions

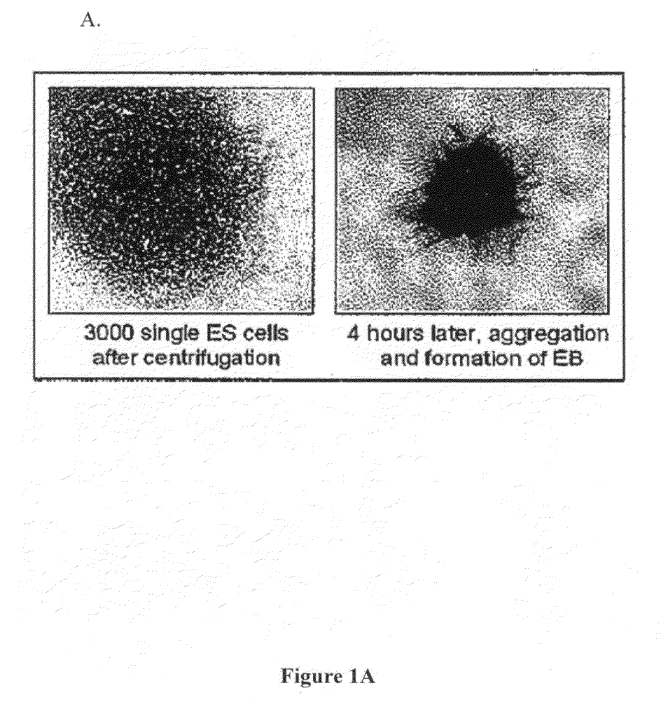

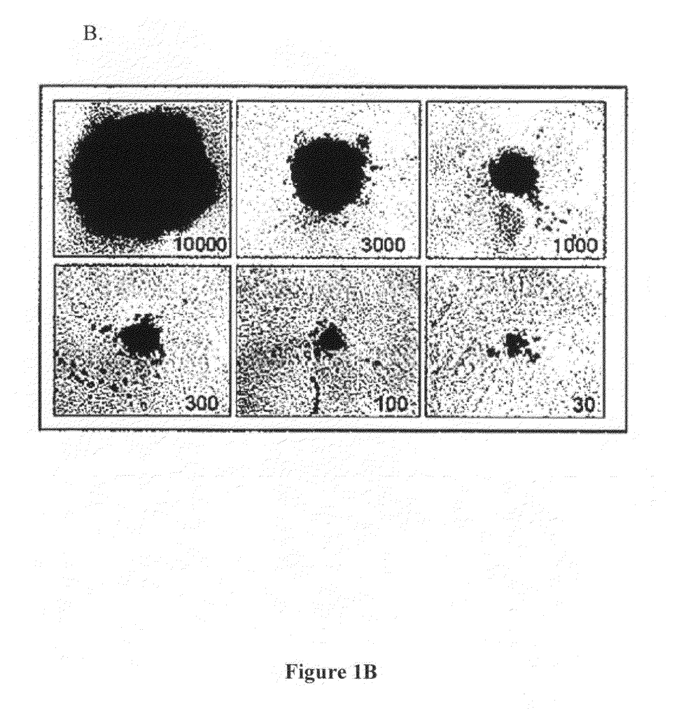

We found that using centrifugation to form hEB from single cell suspensions of hES cells is only successful when murine embryonic fibroblasts (MEF) are mixed with hES cells in the cell pellet. Our investigations have shown that two elements when added to the hESC suspensions are sufficient to allow re-aggregation in the absence of MEF or other feeder cells. These two elements are the extracellular matrix proteins laminin and nidogen (Table 1 and FIG. 1A).

TABLE 1
ES cell aggregation under different conditions (− means no aggregation,
+ means very little aggregation to +++++ meaning
very strong aggregation).

| Inducers of ES cell aggregation | Aggregation |
|---|---|
| Media only, control | − |
| MEF (10% of total cell number in EB) | +++ |
| Human fibroblasts (10% of total cell number in EB) | +++ |
| Media conditioned with MEF | ++ |
| Basal membrane mix (Matrigel), 0.1 mg/ml | +++++ |
| Laminin, collagen IV, nidogen, heparansulphateglycan | |
| Collagen IV, 0.1 mg/ml | − |
| Laminin, 0.1 mg/ml | + |
| Fibronectin, 0.1 mg/ml | − |
| Laminin, 0.1 mg/ml + collagen IV, 0.1 mg/ml | + |
| Laminin/nidogen mix, 0.1 mg/ml | +++++ |
| Laminin/nidogen mix, 0.1 mg/ml + fibrinogen 4 mg/ml | ++ |

By adding human laminin and nidogen complex into suspensions of pure hES cells, cells or products of non-human origin can be completely avoided in the re-aggregation of hESC and the subsequent generation of EB. Laminin binds to the surface of hES cells through the specific receptor integrin α6 β1. The formation of three-dimensional EBs is significantly enhanced by adding a linker molecule that can link laminin molecules, nidogen. Nidogen cross-links large molecules of laminin attached to the hES cell receptors into complex three-dimensional structures. When a laminin-nidogen mix is added to a hES cell suspension, highly viable EBs can be generated from as little as 100 hES cells (FIG. 1B).

Using our method, undifferentiated hES cells are prepared as a single cell suspension, plated (at the desired number of cells) into low attachment round bottom 96 well plates and then re-aggregated to form EBs using centrifugation in the presence of the laminin-nidogen complex. hEBs formed in this way have excellent viability, are of uniform size and contain all three germ layers (ectoderm, mesoderm and endoderm)—that can in turn be induced to generate more differentiated tissue. The simplicity of this method of hEB generation reduces the variability inherent in culture systems that require cellular components (such as MEFs) or other complex media. The simplicity of the system also facilitates the testing of variables that affect differentiation of hEB into different tissue types and improves the quantitation of any effects seen. This system may also be used for generation of chimeric EBs when different cell types (e.g., hES cells transduced with fluorescent tag can be mixed with non-transduced hES cells and re-aggregated into one EB with subsequent tracking of progenies arising from the labeled cells.

### Example 2

### Identification of the Critical Extracellular Matrix Proteins that Promote Human Embryonic Stem Cell Assembly

2.1 Abstract

Human Embryonic Stem Cells (hESC) exist as large colonies containing tightly adherent, undifferentiated cells. Disaggregation of hESC as single cells significantly affects their survival and differentiation, suggesting that adhesion mechanisms are critical for the assembly and maintenance of hESC colonies. The goal of these studies was to determine the key extracellular matrix (ECM) components that regulate assembly and growth of hESC colonies. Our studies demonstrate that undifferentiated hESC express a specific subtype of laminin (laminin-511) and nidogen-1. The addition of a purified protein complex comprised of human laminin-511 and nidogen-1 to single cell suspensions of hESC is sufficient to restore hESC assembly in the absence of murine embryonic fibroblasts or exogenous chemicals. The mechanism of hESC aggregation is through binding of the α6β1 integrin receptor highly expressed in the membranes of undifferentiated hESC; aggregation can be inhibited by an antibody against α6 and almost completely blocked by an antibody against the β1 subunit. Re-assembly of defined numbers of purified hESC with the laminin-nidogen complex allows consistent production of uniform EBs (“LN-EBs”) that differentiate into endodermal, ectodermal and mesodermal derivatives, and are highly efficient in generating hematoendothelial progenitors. These data reveal for the first time the crucial role of the ECM proteins laminin-511 and nidogen-1 in hESC assembly, and provide a novel practical tool to investigate hESC differentiation in a xenogen-free microenvironment.

2.2 Introduction

Embryonic Stem Cell (ESC) lines are generated from cells extracted from the inner cell mass of the blastocyst, and represent in vitro, self-renewing, sources of pluripotent stem cells that offer unique experimental insights in developmental biology and may be potentially developed as a clinical source of transplantable tissue. The ability to expand human ESC (hESC) indefinitely in an undifferentiated state, requires the culture of these cell lines as tightly adherent colonies in contact with established “feeder layers” such as mouse embryonic fibroblasts (MEFs) or in pooled extra-cellular matrix (ECM) products such as Matrigel(1). Disaggregation of hESC as single cells significantly affects their survival and differentiation in culture, suggesting that adhesion mechanisms are critical for the assembly and maintenance of hESC colonies (2).

Directed differentiation of ESCs to tissue specific cells is typically achieved through the generation of embryoid bodies (EBs) which recapitulate many aspects of early embryogenesis (3, 4). The standard process by which EBs are formed begins with the removal of aggregates of ESCs from feeder layers and their transfer as intact clumps into low attachment plates, in which, after formation of three-dimensional EBs, germ-layer differentiation is rapidly induced and terminally differentiated tissue-specific cells are generated. Analysis of this process shows that both the size of the ESC aggregates at the initiation stage, and the methodology used for EB formation, significantly affect subsequent growth and differentiation (2, 5). Despite the clear importance of cell aggregation in the biology and in the experimental manipulation of ESCs and EBs, the precise mechanisms regulating hESC aggregation and EB formation have not been determined.

The assembly of multi-cellular units into complex, three-dimensional tissues requires structural and functional connections to be formed between stromal and epithelial cells through ECM proteins, such as laminins, collagens, nidogens and heparan sulphate proteoglycans (HPSG), located in the basement membranes. During early embryonic development, certain components of the ECM are expressed by cells within the inner cell mass of the blastocyst (6). The goal of these studies was to determine the critical ECM components that regulate assembly and growth of hESC colonies. We also hypothesized that the same ECM molecules play a critical role in the process of EB formation.

2.3 Materials and Methods

2.3.1 Human Embryonic Stem Cell Cultures

hESC lines H1 and H9, obtained from WiCell (Madison, Wis.), were maintained on irradiated primary MEFs in media recommended by WiCell, routinely characterized and found to have a normal karyotype and expression of pluripotency markers SSEA4, CD9, OCT4 and alkaline phosphatase (data not shown). 2.3.2 Immunocytochemistry.

For immunochistochemical analysis, colonies of undifferentiated hESC or EBs were fixed in 4% paraformaldehyde (Sigma-Aldrich, St. Lois, Mo., in PBS) and further processed for immunostaining as described before (7). The primary antibodies were polyclonal antibodies against collagen type IV (ab6586, 1:100; Abcam Inc. Cambridge, Mass.); laminin (ab11575, 1:50; Abcam Inc.); and nidogen (sc-33141, 1:500; Santa Cruz Biotechnology); as well as a monoclonal antibody against heparan sulphate proteoglycans (HSPG; ab2501, 1:100; Abcam Inc.). Secondary antibodies included Alexa Fluor 488-conjugated goat anti-rabbit IgG (H+L) and anti-rat IgG (H+L) (1:250; all from Molecular Probes). To visualize the F-actin cytoskeleton, cells were stained using Alexa Fluor 594 phalloidin (1:40; Molecular Probes). For counterstaining of cell nuclei 4′-6-diamidino-2-phenylindole (DAPI; Sigma-Aldrich) was added to the final PBS washing. Staining without primary antibodies served as controls. Images were acquired using a confocal TCS SP2 AOBS laser-scanning microscope system (Leica Microsystems Inc., Exton, Pa.) with 40× (1.3 numerical aperture (NA)) and 63× (1.4 NA) oil-immersion objectives.

For analysis of hESC differentiation, EBs were removed from low attachment plates, fixed in 4% paraformaldehyde (PFA, Sigma-Aldrich, St. Lois, Mo., in PBS) for 3 hours at room temperature, washed in PBS and embedded in paraffin (McCormic Scientific, St. Lois, Md.). Sections were then deparaffinized, hydrated and immunostained using primary antibodies raised against nestin (mab-1259, R&D systems), alpha-fetoprotein (sc-51506, Santa Cruz Biotechnology, Santa Cruz, Calif.) and CD34 (ab-8536, Abcam Inc., Cambridge, Mass.); followed by washing with PBS containing 0.05% Tween 20 (Sigma-Aldrich) for 3 times, and incubation with secondary anti-goat or anti-mouse antibodies (ImmPRESS; Vector Laboratories, Burlingame, Calif.), with subsequent exposure to an AEC substrate (Santa Cruz Biotechnology). Images were captured with a bright field Image Zeiss light microscope (Carl Zeiss MicroImaging Inc., Thornwood, N.Y.).

2.3.3 Semiquantitative RT-PCR.

Total RNA was extracted from cells using the RNeasy Mini Kit, converted to cDNA using the Omniscript RT Kit, and cDNA subsequently subjected to multiplex PCR performed with Hot Start Master Mix Kit (all kits were from Qiagen Sciences, Maryland, USA) for the evaluation of the mRNA expression of genes of interest. PCR products were resolved by electrophoresis on 2% or 1.5% agarose gels containing ethidium bromide. Gels were scanned using the ImageStation 2000R (Eastern Kodak Company, Rochester, N.Y.) and the bands were analyzed using Kodak 1D analysis software. RT-PCR conditions were 95° C. for 10 min: 1 cycle; 95° C. for 1 min; 55° C. for 90 sec; 72° C. for 1 min: 35 cycles; and 72° C. for 10 min; 1 cycle. RT-PCR primer sequences can be found in Table 2.

TABLE 2
Specific Primer Sequences Used in the RT-PCT Analysis

| | GenBank | | | Amplicon |
|---|---|---|---|---|
| Gene | accession | 5′ Primer | 3′ Primer | size, bp |
| LAMA1 | NM_005559 | GTCAGCGACTCAGAGTGTTTG | AACTTGGGTGAAAGATCGTCAG | 185 |
| LAMA2 | NM_000426 | GAACCCGCAGTGTCGAATCT | GGGGAGTTAGCTGCCTTCA | 204 |
| LAMA3 | NM_000227 | TAGACTTTGGAAGCACCTACTCA | GTTTATCAAGGACACCACAACCT | 185 |
| LAMA4 | NM_002290 | GCAGTGGAAATTCAGATCCCA | TAACCGCAGGTCATCAGTCAG | 275 |
| LAMA5 | NM_005560 | GGTGTGTCTCTGCGTGACAA | CCCCGACGTAGAAGACGAA | 253 |
| LAMB1 | NM_002291 | AGGAACCCGAGTTCAGCTAC | CACGTCGAGGTCACCGAAA | 103 |
| LAMB2 | NM_002292 | GCCCTGGGAACTTCGACTG | GGAAGCACTTCTTTTCGTCCTG | 227 |
| LAMB3 | NM_000228 | TCCTCTTGTGTTTTGCCCTG | CTGCCTGGAGTCACACTTG | 206 |
| LAMC1 | NM_002293 | TCGTCAACGCCGCTTTCAA | GTGTCGGCCTGGTTGTTGTA | 184 |
| LAMC2 | NM_005562 | CCAGGAGGGAAGTCTGTGATT | GCAGTGAATCCCATCAGTGTT | 128 |
| LAMC3 | NM_006059 | CCAGGTGCATCACATCCTGAG | GACCCATTTGGGCTCCATT | 236 |
| NID1 | NM_002508 | CCTGGAGGGAAATACCATGAGG | TCACAFGCAAAAGGATACTGGAGC | 596 |
| NID2 | NM_007361 | TCCTGTACCGAGAGGACACC | TCAGACCCATCAGATGCCAAA | 211 |
| COL4A1 | NM_001845 | CAAAAGGGTGATACTGGAGAACC | ATTTCCTGCGAAACCAGGCA | 210 |
| COL4A2 | NM_001846 | TTGGCGGGTGTGAAGAAGTTT | CCTTGTCTCCTTTACGTCCCTG | 178 |
| COL4A3 | NM_031366 | AGCAAGGGTTGTGTCTGTAAAG | AAGTCCGTAAGGCCCGGTATT | 276 |
| COL4A4 | NM_000092 | AGAGATTGCTCTGTTTGCCAC | CGGTCCCCTCTCATTCCTT | 143 |
| COL4A5 | NM_033380 | CTCCTGGACTTGACGGACAG | TGTCACCTTTCACTCCTTGTTC | 168 |
| COL4A6 | NM_033641 | CTCCTTGCCCTCACTCATAGC | GTCTCCCTTAGGCCCTTTAGG | 181 |

2.3.4 hEB Formation Using Standard and “Spin” EB Approaches.

To generate MEF-free EBs, a modification of the “spin EB” method was used (8, 9). Colonies of H1 and H9 hESC (WiCell Inc) (passage 36-50) were cut into uniformed sized pieces using StemProEZPassage tool, transferred onto 6-well plates coated for 1 hour with Matrigel (growth factor reduced, no phenol red; BD Bioscience), and cultured in MEF-conditioned hESC media supplemented with 4 ng/ml FGF2. Pre-culture on Matrigel increases aggregation capacity of hESC (data not shown). After 24 hours, colonies were digested with Tryple Select into a single cell suspension, incubated with SSEA4 antibody, and SSEA4 positive cells sorted with FACS as described above. Before the sorting step 2-3% of the cells in suspension were represented by MEFs and 97-98% by hESC. After sorting, purity of hESC in the preparation was >99.9% as was measured with SSEA4 staining by FACS. These purified hESC were centrifuged at 1400 RPM and the resulting pellet was resuspended in previously described differentiation media, transferred into low attachment round-bottom 96 well culture plates (Nunc, Rochester, N.Y.) and centrifuged at 1400 RPM for 7 min with or without inducers of aggregation. To test the effects on cell aggregation, a mixture of ECMs in the form of Matrigel (5, 25, 100, 200, μg/ml) a solubilized basement membrane preparation extracted from the Engelbreth-Holm-Swarm (EHS) mouse sarcoma; murine laminin-111 and nidogen-1 complex (5, 25, 100, 200 μg/ml) from EHS sarcoma; or purified human collagen IV (1, 5, 25, 50 μg/ml) (all from BD Bioscience); human laminin-511 (1,5, 25, 50 μg/ml) (Sigma-Aldrich) and human nidogen-1 (1,5, 25, 50 μg/ml) (R&D systems) were added to the cell suspension prior to centrifugation. For directed differentiated of the EBs, differentiation media was supplemented with human vascular endothelial growth factor (VEGF), FGF2, bone morphogenic protein 4 (BMP4), or Wnt3a (all at 10 ng/ml; R&D systems) or FCS (Omega Scientific Inc. Tarzana, Calif.), prior to the final centrifugation step dependent upon experimental design.

“Standard EBs” were generated by collagenase treatment and detachment of clumps of hESC colonies from MEF co-cultures using a well-established protocol(3), and cultured in low-contact plates in the same EB media as described above.

2.3.5 Antibody Staining and Flow Cytometry.

For fluorescence-activated cell sorter (FACS) analysis, 8, 15 or 23 day old EBs were dissociated into a single cell suspension with Tryple Select (Invitrogen) and the cells were incubated with monoclonal antibodies recognizing phycoerythrin (PE)-conjugated BB9/CD143; PE/Cy7-conjugated CD34; fluoresceinisothiocyanate (FITC)-conjugated CD31 (all from BD Biosciences); allophycocyanin (APC)-conjugated KDRNEGFR2 and SSEA4 (R&D systems). For analysis of laminin receptors, cells were incubated for 30 minutes with antibodies against intergrin α3 (Billerica, Mass., USA); α6, β1, and β4 subunits (all from BD Biosciences) and anti-B-CAM (R&D systems). After incubation, cells were washed in PBS containing 1% bovine serum albumin, and analyzed using a BD FACSAria cytometer (BD Bioscience). FACS files were exported and analyzed using FACSDiva software (BD Biosciences) or with ImageStream imaging cytometer using IDEAS software (Amnis Corporation, Seattle, Wash.).

2.3.6 Statistics

Descriptive statistics were performed for each data set and the data combined for collective analysis. Graphs were plotted and data were transformed with Microsoft Excel 2003 (San Diego, Calif.). Statistical analysis was performed with SigmaStat software (Systat Software Inc., Richmond, Calif.). For hematoendothelial progenitor generation studies one-way ANOVA was applied followed by Student-Newman-Keul's test. P<0.05 was considered to be significant.

2.4 Results.

2.4.1 Expression of ECM Proteins in hESC Cultures.

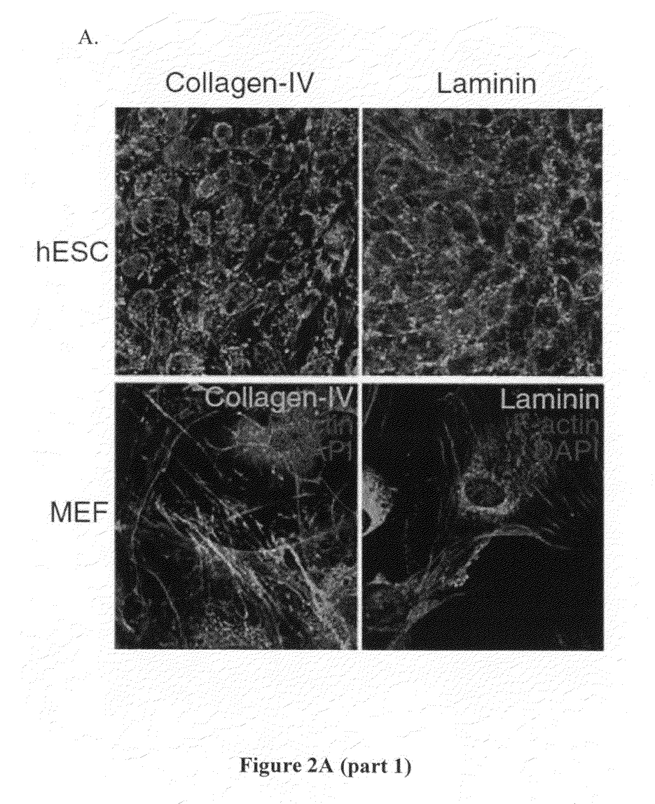

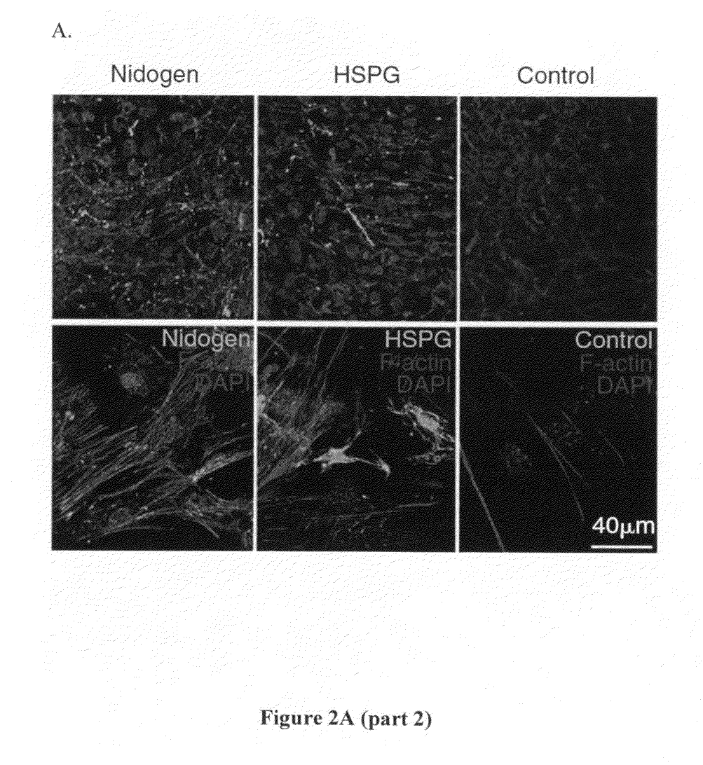

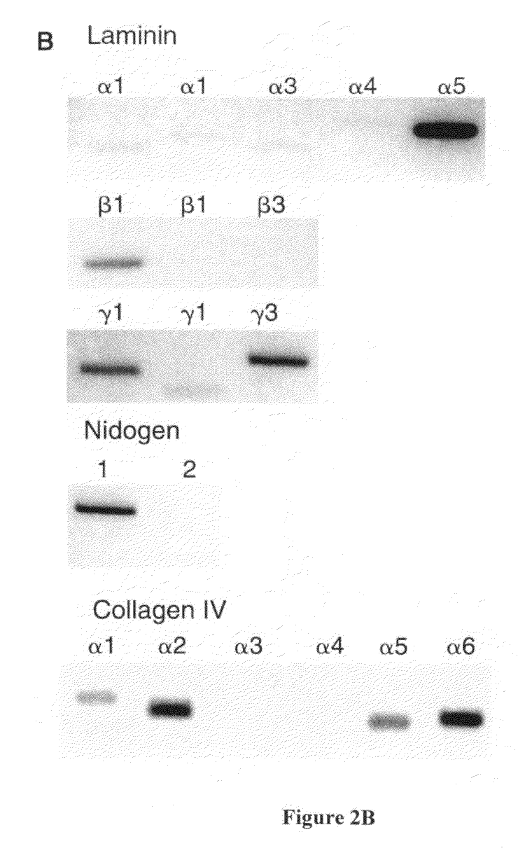

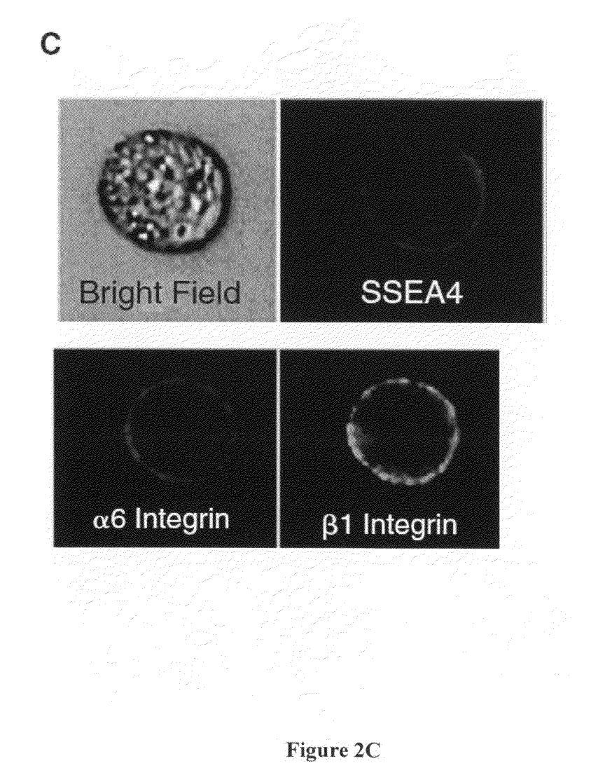

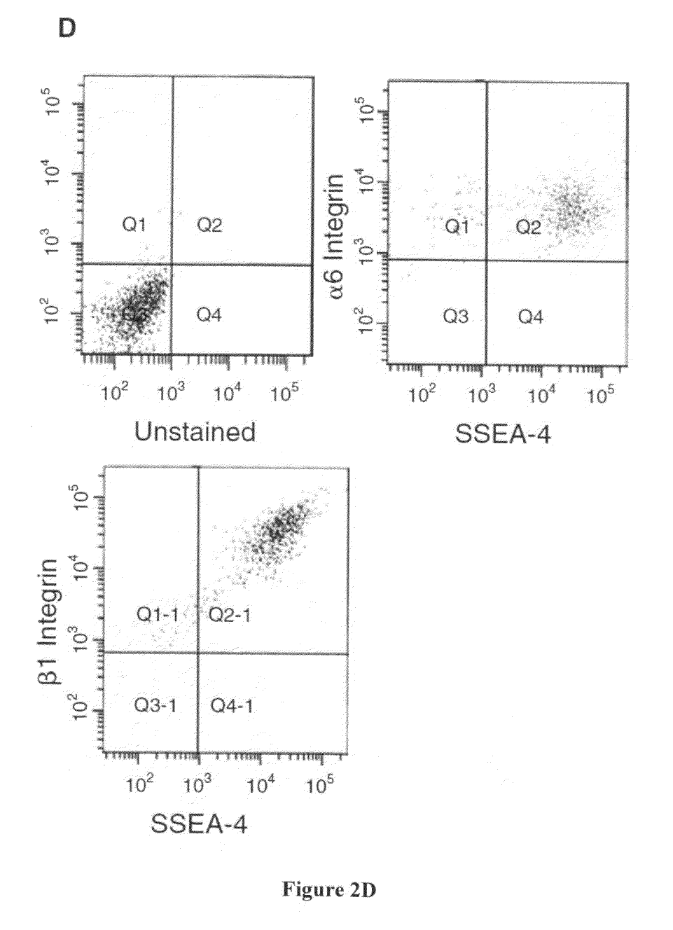

To explore the role of ECM proteins in hESC colony aggregation, we began by analyzing the expression of the key ECM components laminin, collagen IV, HSPG and nidogen in co-cultures of hESC and MEF by fluorescence immunostaining. High levels of collagen IV and laminin expression were seen in the intercellular regions throughout undifferentiated hESC colonies (FIG. 2A). Nidogen protein expression was also present though less prominent, with variable intensity of the staining in different regions within hESC colonies. HSPG expression was barely detectable. As expected, MEFs expressed high levels of all tested ECM molecules including collagen-IV, HSPG, laminin and nidogen (FIG. 2A) and, collagen-I and chondroitin sulphate proteoglycan (data not shown).

To determine if ECM protein expression originated from hESC themselves or were products of MEF co-culture, RT-PCR was performed on MEF-free hESC, isolated as SSEA4+ and CD9+, using primers specific for different human laminin subunits, collagen-IV, and nidogen-1 and -2 genes. Both human laminin and nidogen mRNA transcripts, as well as COL4α1, COL4α2, COL4α5 and COL4α6 genes, encoding the type IV collagen polypeptides, were readily detectable in undifferentiated hESC cells (FIG. 2B). Analysis of hESC showed that the laminin chains α5, β1 and γ1 were expressed, demonstrating that laminin-α5β1γ1 (laminin-511) is the major laminin heterotrimer expressed in undifferentiated hESC. Nidogen-1 was also highly expressed in the hESC colonies, while mRNA transcripts for the nidogen-2 mRNA were not detectable (FIG. 2B). PCR transcripts for other laminin chains were almost undetectable at the undifferentiated stage except for the γ3 chain, the interpretation of which is unclear at this time.

2.4.2 Re-Aggregation of hESC Requires Addition of a Laminin-Nidogen Complex

Despite the endogenous expression of ECM in hESC, when hESC were disaggregated into single cell suspension, depleted of MEFs by FACS sorting of SSEA4 positive cells, and then subjected to centrifugation, no re-aggregation or subsequent EB formation was seen at any time point studied up to 48 hours. Addition of MEFs into the single cell suspension of hESC, at a ratio as low as 1:10 (MEF:hESC) stimulated rapid aggregation of hESC after centrifugation, with EB formation by 24 hours. The partial aggregation was also observed when hESC were centrifuged in the presence of MEF-conditioned media suggesting that the murine feeder layer might provide soluble signals critical for hESC colony assembly.

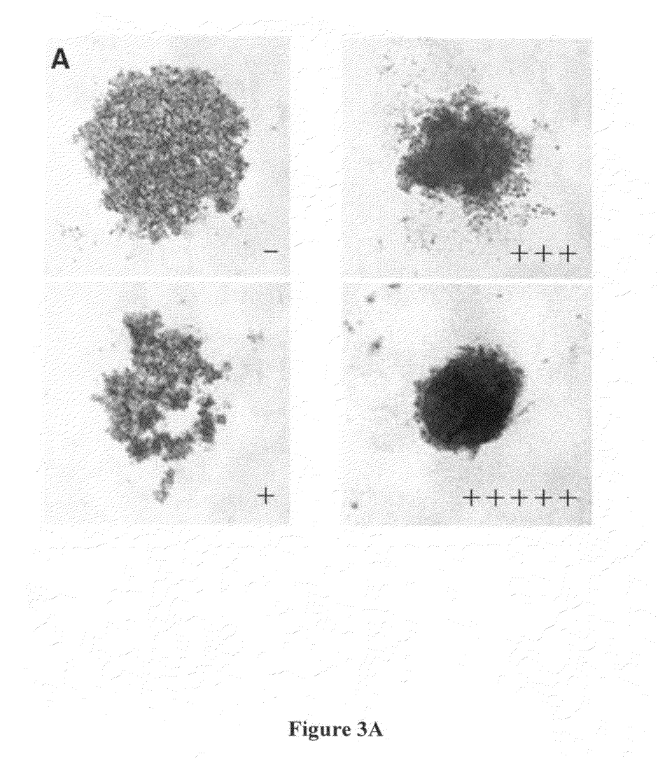

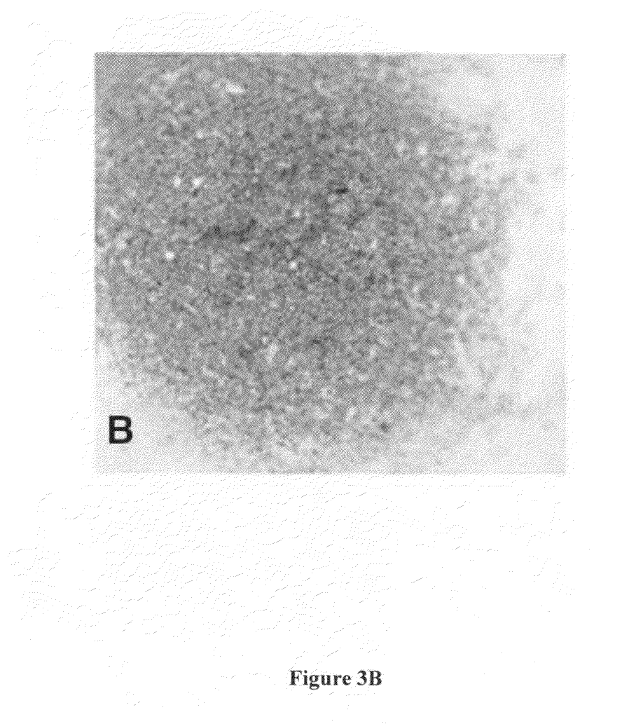

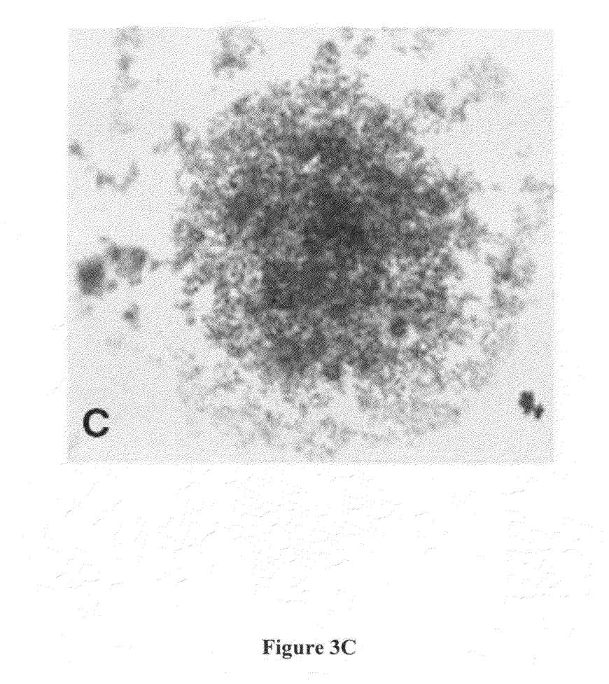

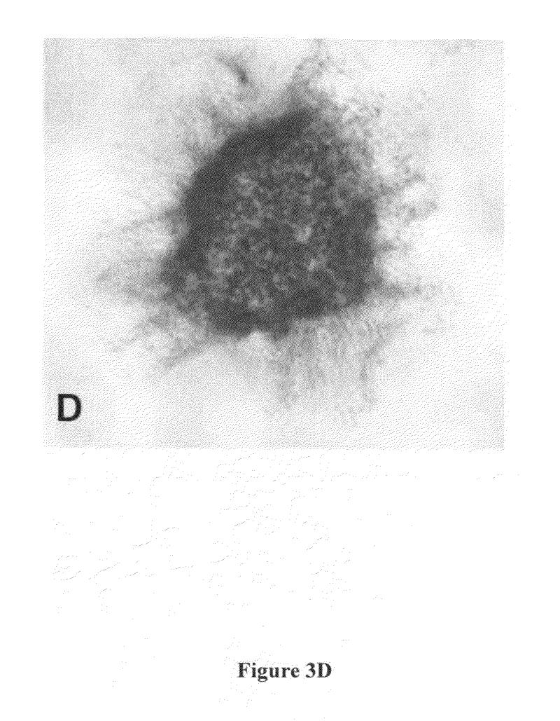

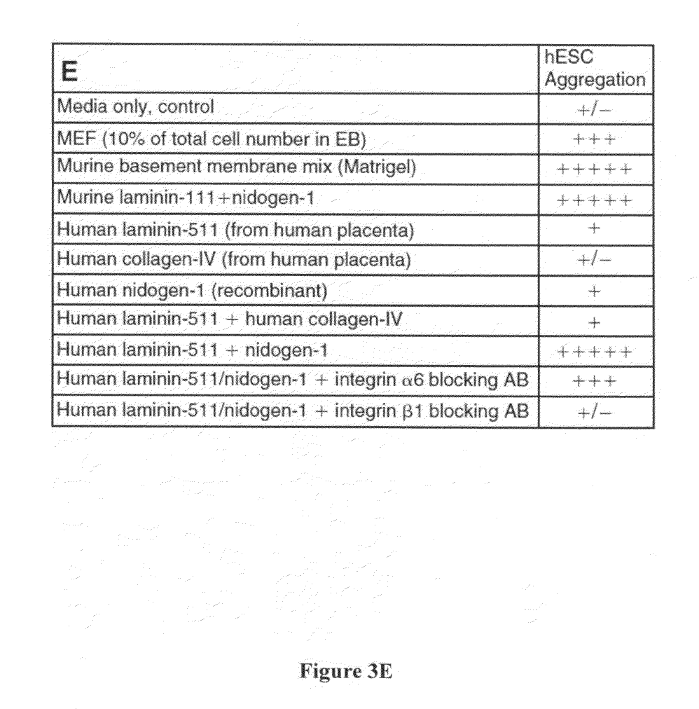

The addition of Matrigel fat final protein concentration 100-200 μg/ml), a combination of murine ECM proteins including laminin-111, collagen-IV, nidogen-1 to a single cell suspension of MEF-free hESC, induced robust cell aggregation clearly detectable within 4 to 8 hours after centrifugation with complete EB formation after 24 hours in culture. Lower concentrations were not sufficient to aggregate all cells in the well. Higher concentrations of Matrigel increased viscosity and blocked efficient pellet formation during centrifugation. The efficiency of cell aggregation under different culture condition was expressed using a semi-quantitative scoring system: “−” representing no cell aggregation, “+++” indicating that 40-60% of cells in the well aggregated and formed EB, while others remain single cells, and “+++++” used when most of the cells in the well aggregated and formed EB after 24 hour in culture (FIG. 3A).

To further dissect the mechanisms of hESC aggregation, the key ECM proteins were tested individually at a range of concentrations with regards to their capacity to assemble dispersed hESC into EBs. No aggregation was seen with human collagen-IV, when added individually to the medium. Only minimal aggregation occurred with the addition of human laminin-511 or nidogen-1 as single agents. However, the application of either murine laminin-111-nidogen-1 complex from EHS tumor (100-200 μg/ml) or ultrapure human laminin-511 (9-22.5 μg/ml) when complexed with human nidogen-1 (1-2.5 μg/ml; ratio laminin to nidogen 9:1), induced robust aggregation of hESC, completely recapitulating the effects of Matrigel (FIG. 3B-E). Thus, the presence of both laminin (either 111 or 511) and nidogen-1 was required for hESC aggregation.

2.4.3 hESC Aggregation is Inhibited by Blocking Adhesion to Laminin Via α6’1 Integrin.

Nidogen acts as a linker between laminins and other ECM molecules essential for the organization of all basement membranes (10). Having established that neither nidogen nor laminin alone are sufficient as single agents, but in combination are highly effective inducing aggregation of hESC, we next tested whether direct hESC binding to the laminin-nidogen complex was required, and explored the receptors involved in such binding. Laminins-111 and -511 bind to cell membranes via the integrin receptors, heterodimers comprised of α and β subunits (11), and also through the basal cell adhesion molecule (B-CAM) (12). Binding of nidogen-1 to integrin receptors expressed in the cell membranes has also been reported (13).

Expression of candidate laminin receptors, on the surface of undifferentiated (SSEA-4+) hESC cultured on Matrigel was tested with imaging flow cytometry technology using specific antibodies against integrins α6, α3, β1 and β4 and basal cell adhesion molecule (B-CAM). α6 and β1 integrin subunits were highly expressed in the plasma membranes of more than 90% of undifferentiated hESC (FIGS. 2C and D). Expression of α3 integrin and B-CAM was highly expressed on 17-19% and 3-5% of analyzed SSEA-4⁺ hESC respectively. Expression of the β4 integrin subunit was almost undetectable in the membranes of undifferentiated (SSEA4-positive) hESC (data not shown). Thus, α6β1 is the dominant integrin expressed on most undifferentiated hESC.

To identify whether binding of hESC to laminin through α6β1 integrin is essential for aggregation, MEF-free hESC suspensions were centrifuged in the presence of human laminin-511-nidogen-1 complex and blocking antibodies to either β1 integrin, α6 integrin, α3 integrin or B-CAM receptor or isotype control antibodies. The addition of blocking antibody against α-6 integrin significantly inhibited and antibody against β1 integrin almost completely abolished cell aggregation (FIG. 3E). In contrast, blocking antibodies against α3 integrin and B-CAM showed minimal or no inhibition of aggregation (data not shown). Thus, despite the fact that laminin alone was not sufficient to induce aggregation, binding of the α6β1 integrin receptor to laminin-511-nidogen-1 complex, was required for hESC aggregation.

2.4.4 “LN-EBs” have Uniform Size and Retain the Capacity for Generating All Three Germ Layers.

We next characterized the EBs formed following laminin-nidogen induced hESC aggregation (“LN-EBs”). LN EBs demonstrated consistent size and growth velocities from both hESC cell lines studied, H1 and H9. LN-EBs were generated from as few as 100 hESC, and at all higher numbers of hESC tested (up to 10,000 cells).

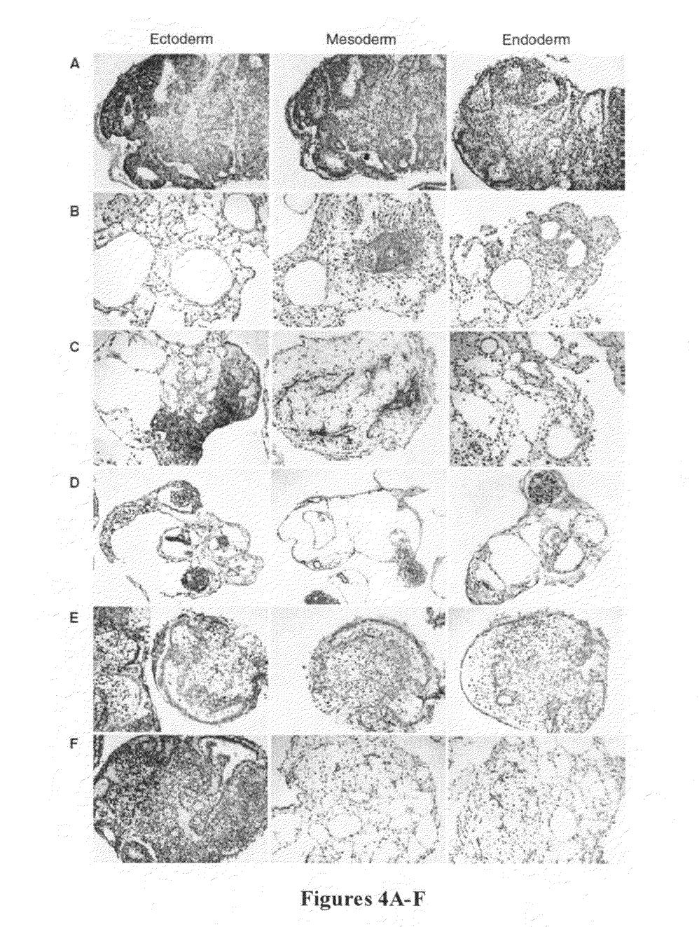

To test the ability of LN-EBs to differentiate into all three germ layers, combinations of different morphogens known to induce formation of ectoderm, mesoderm, or endoderm derivatives in standard EBs, were added to hESC suspension before the centrifugation step and throughout subsequent EB formation (see Experimental Procedures). In serum-free medium, supplemented with Wnt3a, neuro-ectodermal epithelial cells that expressed nestin and formed solid neural tube-like structures were efficiently induced by day 15, and formation of meso-endodermal derivatives labeled with CD34 and alpha-fetoprotein respectively was almost completely abolished (FIG. 4A). In contrast, culture of the LN-EBs in serum-free medium, supplemented with BMP4, FGF2 and VEGF, induced formation of mesodermal and endodermal derivatives but completely blocked the generation of nestin-positive cells (FIG. 4B). When LN-EBs were grown in media supplemented with 10% FCS, all three germ layers were synchronously generated (FIG. 4C). Thus aggregation using the laminin-nidogen complex did not inhibit the germ-layer differentiation potential of hESC.

2.4.5 The “LN-EB” System is Highly Efficient in Generating Hematoendothelial Progenitors.

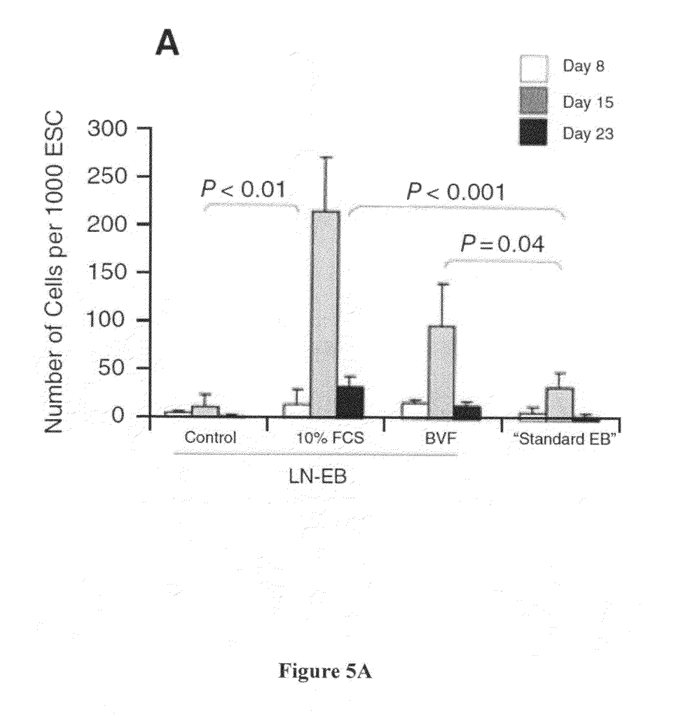

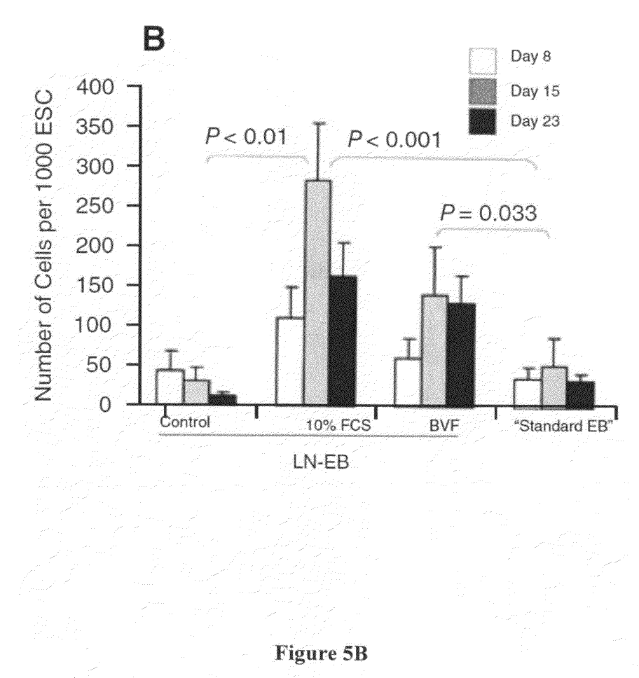

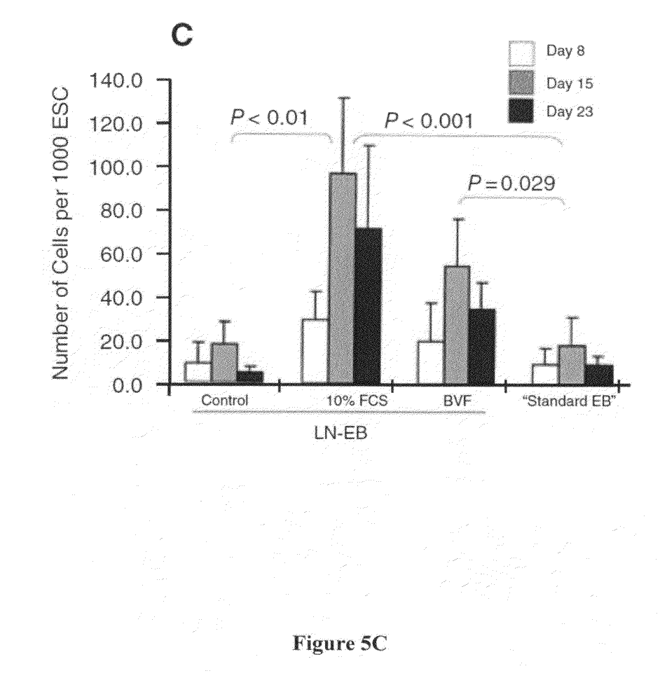

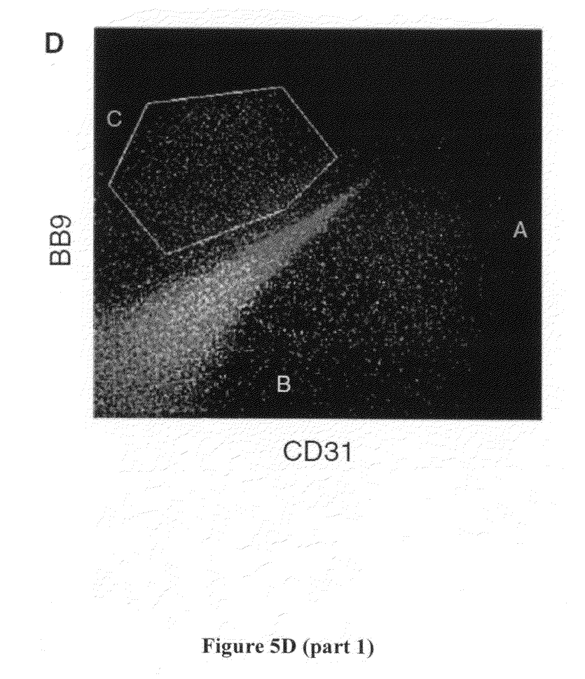

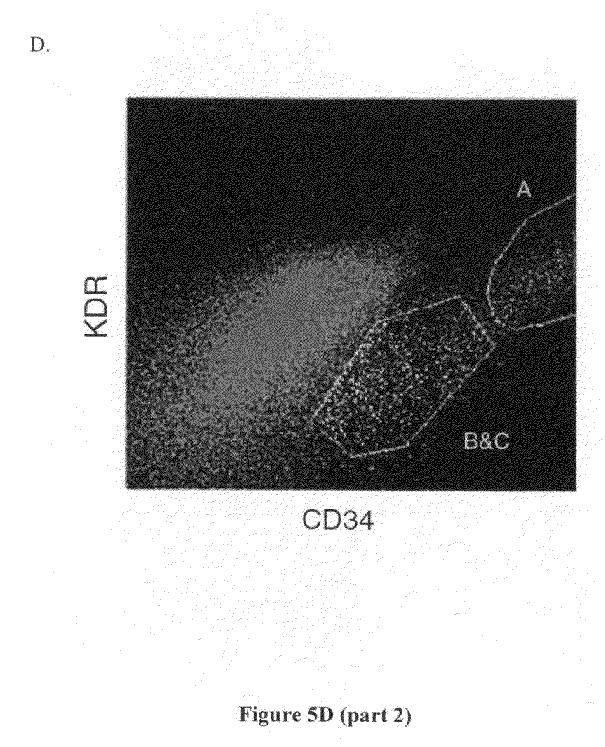

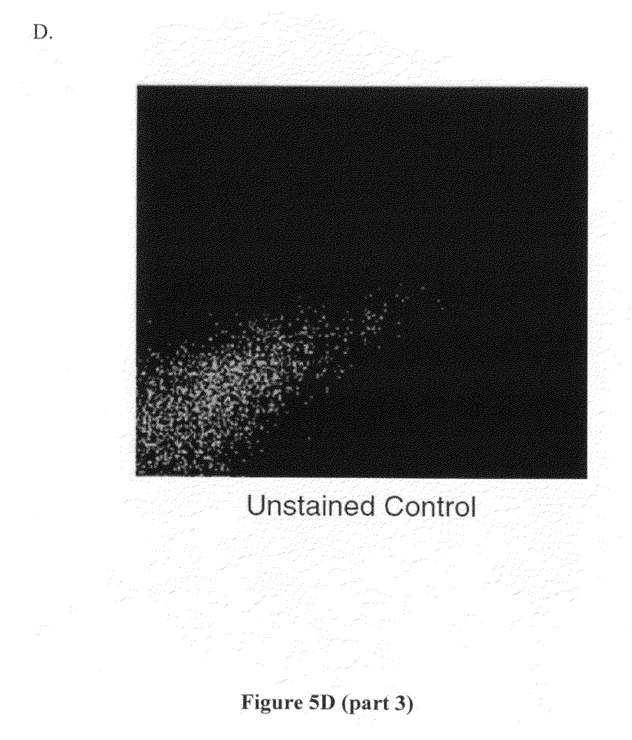

Generation of hematoendothelial progenitor cells from hESC is relatively well characterized and can be used as a model for evaluation of tissue specific differentiation from hESC. For quantitative evaluation of hematoendothelial progenitor cell generation, LN-EBs were compared with “standard EBs”, produced using collagenase detachment of clumps of hESC colonies removed from MEF feeder cells. Using specific markers known to label hematoendothelial cells in vitro and in vivo (14, 15), we identified three major CD34+ sub-populations generated in both EB systems (FIG. 5). Quantitative analysis of hematoendothelial progenitor cell formation within LN-EBs cultured in the presence of 10% FCS for 15 days demonstrated approximately 7 fold more CD34^{bright} CD31^{bright} KDR^{bright} BB9^{dim} cells when compared to those generated from an equivalent number of “standard EBs” (n=5, p<0.01), 5 fold more CD34^{dim} CD31^{dim} KDR_{neg}BB9_{neg} cells (n=5, p<0.001), and 6 fold more CD34^{dim} CD31^{dim} KDR_{neg} BB9^{bright} cells respectively (n=5, p<0.001). The LN-EB system not only produced significantly higher absolute numbers of hematoendothelial progenitors, but intact LN-EBs could be maintained in culture for significantly longer than could EBs formed using standard methods (FIG. 5). Previous studies have shown that CD34 bright cells (Population A in FIG. 5), demonstrate both endothelial and hematopoietic potential; whereas CD34 dim cells (population B), possess predominantly hematopoietic potential (16). The differentiation potential of population C remains unknown and requires future characterization.

2.5 Discussion.

Our studies demonstrate that undifferentiated hESC highly express a specific subtype of laminin (laminin-511) and collagen-IV, and also nidogen-1. The addition of a purified protein complex comprised of human laminin-511 and nidogen-1 to single cell suspensions of hESC is sufficient to restore hESC assembly in the absence of murine embryonic fibroblasts or other exogenous chemicals. This study shows for the first time that hESC express nidogen-1 and reveals functional significance for this protein in hESC colonies.

Nidogen is a linker protein essential for the organization of all basement membranes including those in skin, muscle and the nervous system (17); two nidogen genes have been identified so far (10). Laminins represent a family of heterotrimeric proteins consisting of α, β and γ chains (18). The G3 domain of nidogen-1 binds with high-affinity to a single laminin-type epidermal growth factor-like (LE) module of the γ1 chain, present in laminins α5β1γ1 (511) and α1β1γ1 (111) (19, 20). This binding is thought to be particularly important for basement membrane assembly (21). To the best of our knowledge the expression and role of nidogen-1 in hESC has not been previously reported. In a recently published study, laminin protein expression was shown in HUES1, HES2, HESC-NL3 lines while collagen IV was found exclusively in the mouse feeder area (22). Our data clearly indicate that both H1 and H9 hESC lines highly express COL4α1, COL4α2, COL4α5 and COL4α6 genes, encoding the type IV collagen polypeptides. We also show in this study that collagen type IV is highly expressed in the intracellular regions of undifferentiated H1 and H9 colonies. The exact reason for the discrepancy of these findings is not completely clear, but may reflect functional dissimilarities of different hESC lines. In agreement with previously published reports (1), we showed that the β1 integrin subunit, in combination with the α6 integrin subunit, was highly expressed in the membranes of undifferentiated hESC. This integrin receptor was previously implicated in the laminin-mediated adhesion of hESC (1). Our data clearly showed that α6β1 integrin subunit is also crucial for the re-aggregation of hESC.

Our previous studies demonstrate that some ECM molecules, in particular collagen IV can induce mesodermal differentiation of murine ESC (7). In the current study LN-EBs were shown to efficiently generate all three germ layers in response to differentiation cytokines and morphogens added to the culture media. At the same time, the presence of high concentrations of laminin and nidogen at the initiation stage, markedly enhanced assembly of hESC within LN-EBs leading to a much complex histological composition of these EBs maintained for up to 30 days in culture compared to standard EBs, which undergo cystic transformation and morphological involution from day 7-8 of culture.

Although undifferentiated hESC expressed both laminin and nidogen proteins, when hESC were removed from culture and dispersed into single cell suspension, their immediate re-aggregation required the addition of exogenous ECM proteins. Recently the p160-Rho associated coiled kinase (ROCK) inhibitor Y-27632 has been reported to promote the survival of hESC after dissociation to single cells (23), and improve their ability to form EBs. Other authors have demonstrated that dissociated hESC aggregate and form EBs in presence of 10 μM of Y-27632 (2). Based on these observations it can be speculated that improved survival of hESC may allow them to produce sufficient levels of laminin and nidogen, thus resulting in self-aggregation without exogenous addition of the laminin-nidogen complex. Of note, consistent with previous reports (8, 24), we found that the addition of only small numbers of MEFs or MEF-conditioned media can induced aggregation of hESC after centrifugation, albeit to a lesser extent when compared to ECM protein addition, suggesting that MEFs may also provide either survival factors or ECM molecules that can promote the assembly of hESC.

In summary, our data show that the ECM proteins laminin and nidogen are naturally expressed in undifferentiated hESC colonies and can induce efficient hESC aggregation when added in high concentrations to MEF-free suspensions of hESC. The LN-EBs formed from re-aggregated hESC, are highly viable, uniform in size and recapitulate the properties of the inner cell mass to undergo synchronous differentiation into endodermal, ectodermal and mesodermal derivatives. In comparison to standard EBs, LN-EBs were able to produce higher quantities of hematoendothelial progenitor cells, demonstrating efficient tissue specific differentiation. As with the spin EBs previously described(8), the ability to generate LN-EBs from single cell suspensions allows control of EB size, generation of chimeric EBs using different types of hESC, and quantitative assessment of efficiency in tissue differentiation. However, the laminin-nidogen system represents a significant advance over previous methods of EB formation, as it avoids the need for xenogenic reagents or artificial chemicals to induce aggregation, by employing a combination of purified, defined human ECM proteins that are expressed naturally by undifferentiated hESC colonies.

2.6 References.
1. Xu C, M S Inokuma, J Denham, K Golds, P Kundu, J D Gold and M K Carpenter. (2001). Feeder-free growth of undifferentiated human embryonic stem cells. Nat Biotechnol 19:971-974.
2. Ungrin M D, C Joshi, A Nica, C Bauwens and P W Zandstra. (2008). Reproducible, ultra high-throughput formation of multicellular organization from single cell suspension-derived human embryonic stem cell aggregates. PLoS ONE 3:e1565.
3. Itskovitz-Eldor J, M Schuldiner, D Karsenti, A Eden, O Yanuka, M Amit, H Soreq and N Benvenisty. (2000). Differentiation of human embryonic stem cells into embryoid bodies compromising the three embryonic germ layers. Mol Med 6:88-95.
4. Schuldiner M, O Yanuka, J Itskovitz-Eldor, D A Melton and N Benvenisty. (2000). Effects of eight growth factors on the differentiation of cells derived from human embryonic stem cells. Proc Natl Acad Sci USA 97:11307-11312.
5. Torisawa Y S, B H Chueh, D Huh, P Ramamurthy, T M Roth, K F Barald and S Takayama. (2007). Efficient formation of uniform-sized embryoid bodies using a compartmentalized microchannel device. Lab Chip 7:770-776.
6. Dziadek M and R Timpl. (1985). Expression of nidogen and laminin in basement membranes during mouse embryogenesis and in teratocarcinoma cells. Dev Biol 111:372-382.
7. Schenke-Layland K, Angelis E, Rhodes K E, Heydarkhan-Hagvall S, Mikkola H K and M W R. (2007). Collagen IV induces trophoectoderm differentiation of mouse embryonic stem cells. Stem Cells 25:1529-1538.
8. Ng E S, R P Davis, L Azzola, E G Stanley and A G Elefanty. (2005). Forced aggregation of defined numbers of human embryonic stem cells into embryoid bodies fosters robust, reproducible hematopoietic differentiation. Blood 106:1601-1603.
9. Ng E S, R Davis, E G Stanley and A G Elefanty. (2008). A protocol describing the use of a recombinant protein-based, animal product-free medium (APEL) for human embryonic stem cell differentiation as spin embryoid bodies. Nat Protoc 2008:5.
10. Ho M S, K Böse, S Mokkapati, R Nischt and N Smyth. (2008). Nidogens-Extracellular matrix linker molecules. Microsc Res Tech 71:387-395.
11. Nishiuchi R, J Takagi, M Hayashi, H Ido, Y Yagi, N Sanzen, T Tsuji, M Yamada and K Sekiguchi. (2006). Ligand-binding specificities of laminin-binding integrins: A comprehensive survey of laminin-integrin interactions using recombinant alpha3beta1, alpha6beta1, alpha7beta1 and alpha6beta4 integrins. Matrix Biol 25:189-197.
12. Kikkawa Y, C L Moulson, I Virtanen and J H Miner. (2002). Identification of the binding site for the Lutheran blood group glycoprotein on laminin alpha 5 through expression of chimeric laminin chains in vivo. J Biol Chem 277:44864-44869.
13. Dedhar S, K Jewell, M Rojiani and V Gray. (1992). The receptor for the basement membrane glycoprotein entactin is the integrin alpha 3/beta 1. J Biol Chem 267.
14. Perlingeiro R C, M Kyba, S Bodie and G Q Daley. (2003). A role for thrombopoietin in hemangioblast development. Stem Cells 21:272-280.
15. Jokubaitis V J, L Sinka, R Driessen, G Whitty, D N Haylock, I Bertoncello, I Smith, B Péault, M Tavian and P J Simmons. (2008). Angiotensin-converting enzyme (CD143) marks hematopoietic stem cells in human embryonic, fetal, and adult hematopoietic tissues. Blood 111:4055-4063.
16. Woll P S, J K Morris, M S Painschab, R K Marcus, A D Kohn, T L Biechele, R T Moon and D S Kaufman. (2008). Wnt signaling promotes hematoendothelial cell development from human embryonic stem cells. Blood 111:122-131.
17. Takagi J, Y Yang, J H Liu, J H Wang and T A Springer. (2003). Complex between nidogen and laminin fragments reveals a paradigmatic beta-propeller interface. Nature 424:969-974.
18. Timpl R. (1996). Macromolecular organization of basement membranes. Curr Opin Cell Biol 8:618-624.
19. Mayer U, R Nischt, E Poschl, K Mann, K Fukuda, M Gerl, Y Yamada and R Timpl. (1993). A single EGF-like motif of laminin is responsible for high affinity nidogen binding. EMBO J 12:1879-1885.
20. Poschl E, J W Fox, D Block, U Mayer and R Timpl. (1994). Two non-contiguous regions contribute to nidogen binding to a single EGF-like motif of the laminin gamma 1 chain. EMBO J 13:3741-3747.
21. Gersdorff N, E Kohfeldt, T Sasaki, R Timpl and N Miosge. (2005). Laminin gamma3 chain binds to nidogen and is located in murine basement membranes. J Biol Chem 280:22146-22153
22. Braam S R, L Zeinstra, S Litjens, D Ward-van Oostwaard, S van den Brink, L van Laake, F Lebrin, P Kats, R Hochstenbach, R Passier, A Sonnenberg and C L Mummery. (2008). Recombinant vitronectin is a functionally defined substrate that supports human embryonic stem cell self-renewal via alphavbeta5 integrin. Stem Cells 26:2257-2265.
23. Watanabe K, M Ueno, D Kamiya, A Nishiyama, M Matsumura, T Wataya, J B Takahashi, S Nishikawa, K Muquruma and Y Sasai. (2007). A ROCK inhibitor permits survival of dissociated human embryonic stem cells. Nat Biotechnol 25:681-686.
24. Burridge P W, D Anderson, H Priddle, M D Barbadillo Muñoz, S Chamberlain, C Allegrucci, L E Young and C Denning. (2007). Improved human embryonic stem cell embryoid body homogeneity and cardiomyocyte differentiation from a novel V-96 plate aggregation system highlights interline variability. Stem Cells 25:929-938.

### Example 3

### LN-EBs Produce ECM Proteins.

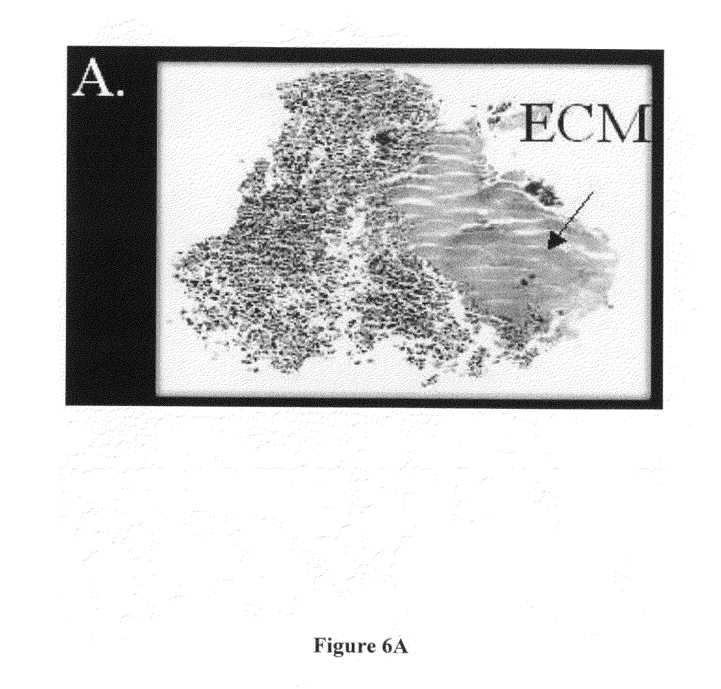

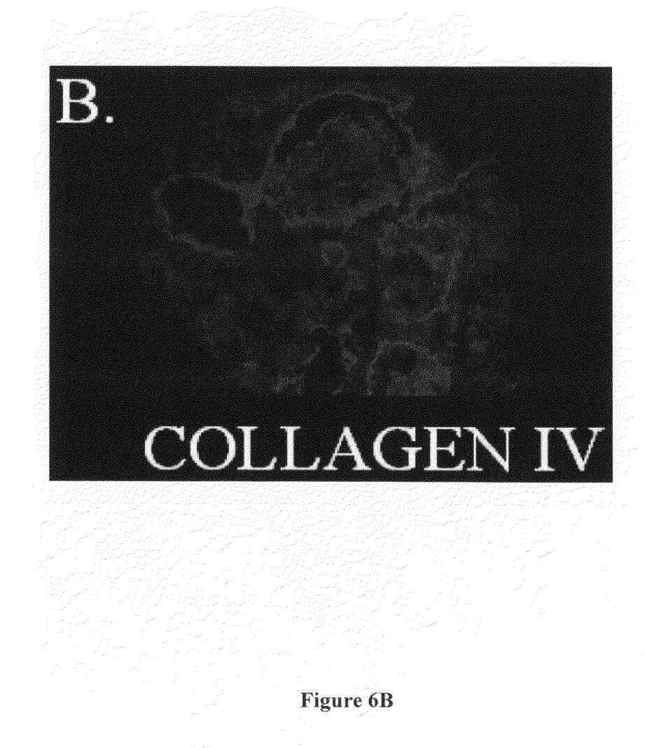

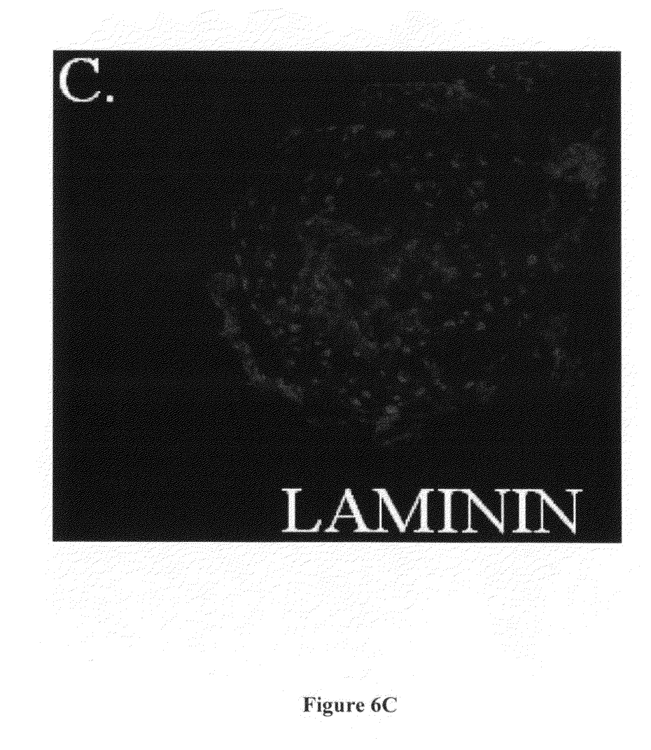

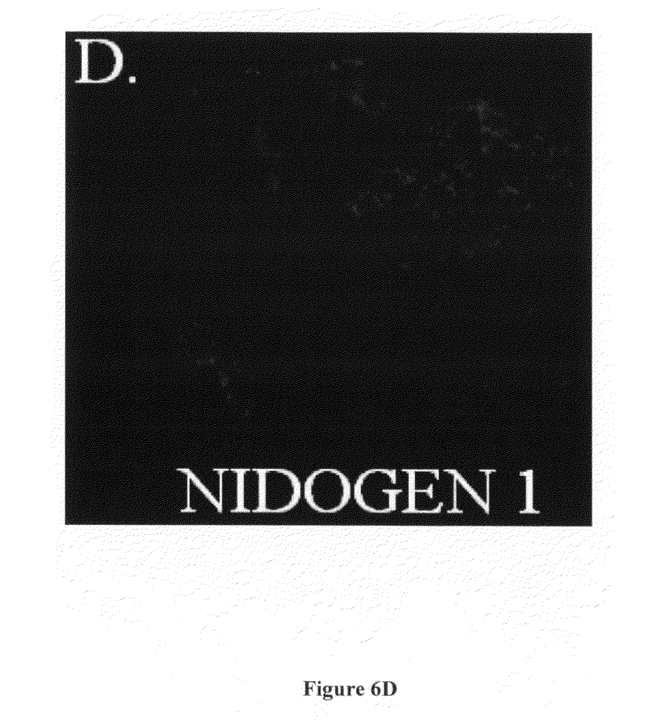

The expression of ECM proteins was examined using antibodies on frozen sections of LN-EBs. LN-EBs produce large amounts of ECM proteins deposited as massive protein islands within the EBs (FIG. 6A), or diffusely distributed within the cellular compartments (FIGS. 6B, 6C, 6D). Frozen sections of LN-EBs were prepared on day 15 of culture and exposed to primary polyclonal antibodies against collagen IV, laminin and nidogen 1, respectively. Then, the sections were exposed to the secondary antibodies conjugated with Cy3 (red color). Nuclei were visualized with Dapi staining (blue).

In accordance to our PCR data suggesting that laminin 511, nidogen 1 and collagen IV are expressed in undifferentiated hESC, these data demonstrated that human LN-EBs generate large amount of these proteins. Human EBs can therefore be used as a source for human laminin 511 and nidogen 1 and collagen IV production and purification.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All cited publications, patents, and patent applications are herein incorporated by reference in their entirety for any purpose.

1. A composition comprising a culture medium for an ES cell; wherein said medium is conditioned with laminin and nidogen; wherein said medium is capable of inducing formation of EBs.

2. The composition according to claim 1 wherein the composition further comprises fibronectin.

3. The composition according to claim 1 wherein the medium is conditioned with laminin and nidogen at a concentration of 0.01-2.5 mg/ml each.

4. The composition according to claim 1 wherein the medium is conditioned with laminin and nidogen at a concentration of 0.1 mg/ml each.

5. The composition according to claim 1 further comprising a number of ES cells sufficient to form an EB.

6. A method for EB formation comprising culturing ES cells with a composition according to claim 1.

7. A composition comprising an EB, wherein said EB was generated by culturing ES cells with a composition according to claim 1.

8. A composition comprising a cell, wherein said cell was derived from an ES cell that was cultured with a composition according to claim 1.


SEQUENCE LISTING (38 sequences)

SEQ ID NO: 1 - DNA, 21 bp, human
gtcagcgactcagagtgtttg

SEQ ID NO: 2 - DNA, 22 bp, human
aacttgggtgaaagatcgtcag

SEQ ID NO: 3 - DNA, 20 bp, human
gaacccgcagtgtcgaatct

SEQ ID NO: 4 - DNA, 19 bp, human
ggggagttagctgccttca

SEQ ID NO: 5 - DNA, 23 bp, human
tagactttggaagcacctactca

SEQ ID NO: 6 - DNA, 23 bp, human
gtttatcaaggacaccacaacct

SEQ ID NO: 7 - DNA, 21 bp, human
gcagtggaaattcagatccca

SEQ ID NO: 8 - DNA, 21 bp, human
taaccgcaggtcatcagtcag

SEQ ID NO: 9 - DNA, 20 bp, human
ggtgtgtctctgcgtgacaa

SEQ ID NO: 10 - DNA, 19 bp, human
ccccgacgtagaagacgaa

SEQ ID NO: 11 - DNA, 20 bp, human
aggaacccgagttcagctac

SEQ ID NO: 12 - DNA, 19 bp, human
cacgtcgaggtcaccgaaa

SEQ ID NO: 13 - DNA, 19 bp, human
gccctgggaacttcgactg

SEQ ID NO: 14 - DNA, 22 bp, human
ggaagcacttcttttcgtcctg

SEQ ID NO: 15 - DNA, 20 bp, human
tcctcttgtgttttgccctg

SEQ ID NO: 16 - DNA, 19 bp, human
ctgcctggagtcacacttg

SEQ ID NO: 17 - DNA, 19 bp, human
tcgtcaacgccgctttcaa

SEQ ID NO: 18 - DNA, 20 bp, human
gtgtcggcctggttgttgta

SEQ ID NO: 19 - DNA, 21 bp, human
ccaggagggaagtctgtgatt

SEQ ID NO: 20 - DNA, 21 bp, human
gcagtgaatgccatcagtgtt

SEQ ID NO: 21 - DNA, 21 bp, human
ccaggtgcatcacatcctcag

SEQ ID NO: 22 - DNA, 20 bp, human
gaccccatttgggctccatt

SEQ ID NO: 23 - DNA, 22 bp, human
cctggagggaaataccatgagg

SEQ ID NO: 24 - DNA, 23 bp, human
tcacagcaaaaggatactggagc

SEQ ID NO: 25 - DNA, 20 bp, human
tcctgtaccgagaggacacc

SEQ ID NO: 26 - DNA, 21 bp, human
tcagacccatcagatgccaaa

SEQ ID NO: 27 - DNA, 23 bp, human
caaaagggtgatactggagaacc

SEQ ID NO: 28 - DNA, 20 bp, human
atttcctgcgaaaccaggca

SEQ ID NO: 29 - DNA, 21 bp, human
ttggcgggtgtgaagaagttt

SEQ ID NO: 30 - DNA, 22 bp, human
ccttgtctcctttacgtccctg

SEQ ID NO: 31 - DNA, 22 bp, human
agcaagggttgtgtctgtaaag

SEQ ID NO: 32 - DNA, 21 bp, human
aagtccgtaaggcccggtatt

SEQ ID NO: 33 - DNA, 21 bp, human
agagattgctctgtttgccac

SEQ ID NO: 34 - DNA, 19 bp, human
cggtcccctctcattcctt

SEQ ID NO: 35 - DNA, 20 bp, human
ctcctggacttgacggacag

SEQ ID NO: 36 - DNA, 22 bp, human
tgtcacctttcactccttgttc

SEQ ID NO: 37 - DNA, 21 bp, human
ctccttgccctcactcatagc

SEQ ID NO: 38 - DNA, 21 bp, human
gtctcccttaggccctttagg